(19) United States
(12) Patent Application Publication
(10) Pub. No.: US 2010/0292736 A1
(43) Pub. Date: Nov. 18, 2010

(54) Linkage for Connection of Fusion and Non-Fusion Systems

(75) Inventor: Frank J. Schwab, New York, NY (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/466,617

(22) Filed: May 15, 2009

(51) Int. Cl. A61B 17/70; A61B 17/88

(52) U.S. Cl. 606/278; 606/279

(57) ABSTRACT

A linkage for connecting together an elongated member of a fusion system and an elongated member of a non-fusion system. The linkage may include a bridge that extends between a first connector and a second connector. The first connector may be configured to connect to a fusion system, and the second connector may be configured to connect to a non-fusion system. The length of the bridge provides for the connectors to be spaced apart by a predetermined length. The linkage may be configured to position the elongated members in various orientations.

### BACKGROUND

Spinal fusion and non-fusion systems may be implanted along different sections of a patient's spine. Fusion systems immobilize two or more vertebral members, often to eliminate pain caused by motion of the vertebral members. Conditions for which spinal fusion may be performed include degenerative disc disease, vertebral fractures, scoliosis, or other conditions that cause instability of the spine. One type of spinal fusion fixes the vertebral members in place with hardware such as hooks or pedicle screws attached to elongated members such as rods on one or each lateral side of the vertebral members. Often, the fusion system is used in combination with a bone graft between the transverse processes or other vertebral protrusions or bone surfaces. The bone graft may rely on supplementary bone tissue, biological agents, and bone growth stimulators in conjunction with the body's natural bone growth processes to literally fuse vertebral members to one another.

Non-fusion systems include a flexible or articulated elongated member such as a tether, ligament, cable, and flexible rod that is connected to two or more vertebral members. The non-fusion system does not usually fully or completely immobilize the vertebral members, but rather applies a force to a section of the spine to align the vertebral members or maintain a range of motion in order to treat various conditions including deformities affecting the normal alignment and curvature of the vertebral members. Scoliosis is one example of a deformity of the spine in the coronal plane, in the form of an abnormal curvature. The types of scoliotic deformities include thoracic, thoracolumbar, lumbar or can constitute a double curve in both the thoracic and lumbar regions. Scheuermann's kyphosis is another example of a spinal deformity that affects the normal alignment of the vertebral members.

A linkage should be available to connect the elongated members of fusion and non-fusion systems where the elongated members are connected to different sections of the spine.

### SUMMARY

The present application is directed to linkages and methods of connecting fusion and non-fusion systems wherein the fusion system is connected to a first section of the spine and the non-fusion system is connected to a second section of the spine.

The linkages may include an elongated bridge with a first end and a second end. A first connector may be connected to the first end of the bridge to connect with the elongated member of the fusion system. The first connector may include a receiver configured to contact against the exterior surface of the elongated member. A second connector may be connected to the second end of the bridge to connect with the elongated member of the non-fusion system. The second connector may include a second connector with opposing surfaces that contact against an outer surface of the elongated member.

The linkages may connect the elongated members in various orientations. The linkages may connect these members in an end-to-end orientation with the systems extending along different sections of the spine, or the linkages may connect the members in an overlapping orientation.

The second connector may be configured to maintain the non-fusion elongated member under tension. The connector may include opposing and adjustable contact surfaces. The contact surfaces may be movable between a first open orientation that allows for the elongated member to be inserted into the second connector, and a second closed orientation with the contact surfaces contacting against the elongated member and preventing the elongated member from moving out of the second connector.

The various aspects of the various embodiments may be used alone or in any combination, as is desired.

### BRIEF DESCRIPTION OF THE DRAWINGS

FIG. 1 is a perspective view of a linkage according to one embodiment.

FIG. 2 is a schematic side view of a linkage connecting a fusion system to a non-fusion system according to one embodiment.

FIG. 3 is a perspective view of a first connector and a section of a bridge according to one embodiment.

FIG. 4 is a perspective view of a first connector and a section of a bridge according to one embodiment.

FIG. 5 is a perspective view of second connector and a section of a bridge according to one embodiment.

FIG. 6 is a perspective view of second connector according to one embodiment.

FIG. 7 is a perspective view of a second connector according to one embodiment.

FIG. 7A is a schematic cross-section taken along line 7A-7A of FIG. 7.

FIG. 8 is a perspective view of a second connector according to one embodiment.

FIG. 8A is a cross-section taken along line 8A-8A of FIG. 8.

FIG. 9 is a schematic view of a bridge extending between first and second connectors according to one embodiment.

FIG. 10 is a cross section of an anchor for connecting an elongated member to a vertebral member according to one embodiment.

FIG. 11 is a schematic side view of a linkage connecting an elongated member of a fusion system and an elongated member of a non-fusion system according to one embodiment.

FIG. 12 is a schematic diagram of a linkage connecting together elongated members of fusion and non-fusion systems.

### DETAILED DESCRIPTION

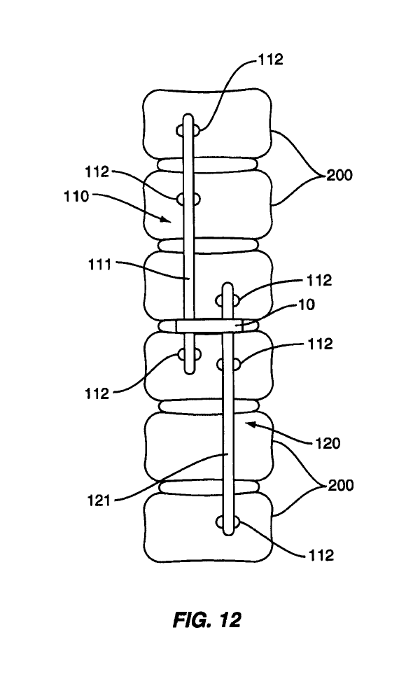

The present application is directed to linkages for connecting together fusion and non-fusion systems. FIG. 12 schematically illustrates a linkage 10 that connects a hybrid system that includes a fusion system 110 that extends along vertebral members 200 of a first section of the spine with a non-fusion system 120 that extends along vertebral members 200 of a second section of the spine. In this embodiment, the systems 110, 120 overlap along a section of the spine, although other embodiments (e.g., FIG. 2) include non-overlapping systems 110, 120. The connection of the non-fusion system 120 to the fusion system 110 may protect against junctional failure, risk of accelerated degeneration, and kyphosis at spinal levels beyond the section of the spine that is directly treated by the fusion system 110.

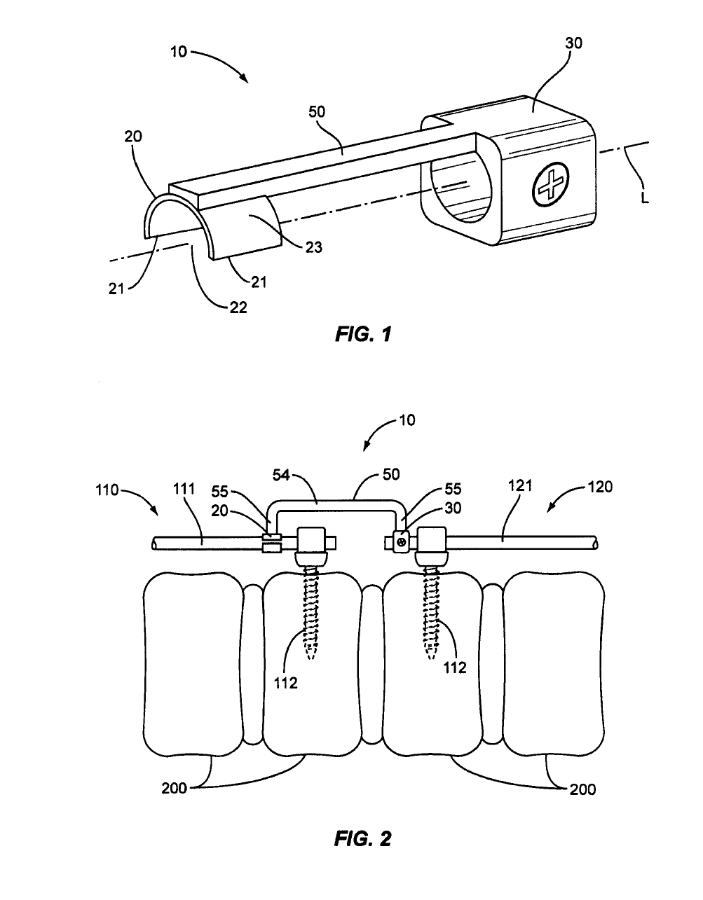

FIG. 1 illustrates a linkage 10 that generally includes a bridge 50 that extends between a first connector 20 and a second connector 30. The first connector 20 is configured to connect to a fusion system 110, and the second connector 30 is configured to connect to a non-fusion system 120. The length of the bridge 50 provides for the connectors 20, 30 to be spaced apart by a predetermined length. FIG. 1 includes the connectors 20, 30 aligned along a longitudinal axis L.

FIG. 2 schematically illustrates the linkage 10 connecting a fusion system 110 and a non-fusion system 120. In many instances, the fusion system 110 includes a rigid elongated member 111. The non-fusion system 120 includes a deformable or articulated elongated member 121 such as but not limited to a tether, artificial ligament, cable, and flexible rod. The connectors 20, 30 are configured to provide secure attachments to these types of structures. The elongated member 111 of the fusion system 110 is connected to a first section of vertebral members 200 with one or more anchors 112. The elongated member 121 of the non-fusion system 120 is connected to a second section of vertebral members 200 with one or more anchors 112. The connector 10 bridges between and connects the systems 110, 120 together. The first connector 20 connects to the elongated member 111 of the fusion system 110, and the second connector 30 connects to the elongated member 121 of the non-fusion system 120. FIG. 2 includes the linkage 10 shaped to connect the elongated member 111 and elongated member 121 in an end-to-end and non-overlapping orientation. The bridge 50 and/or connectors 20, 30 may also be positioned to accommodate the members 111, 121 in an overlapping orientation.

The first connector 20 may include a variety of structural configurations to connect to the elongated member 111 of the fusion system 110. FIG. 1 includes the first connector 20 with a body 23 with a pair of opposing fingers 21 separated by a gap 22. The fingers 21 are flexible with an exterior force elastically deforming the fingers 21 apart to increase the size of the gap 22 and receive the elongated member 111. The fingers 21 may then return towards their original position once the force is removed with the fingers 21 compressing against the elongated member 111 to form the connection. The size of the fingers 21 and gap 22 may vary. FIG. 1 includes the fingers 21 forming a substantially 180° semicircle. The fingers 21 may include different sizes to extend different amounts around the elongated member 111. FIG. 1 includes fingers 21 being curved to match the shape of a circular cross-section elongated member 111. The fingers 21 may also include different shapes that may or may not match the cross-sectional shape of the elongated member 111.

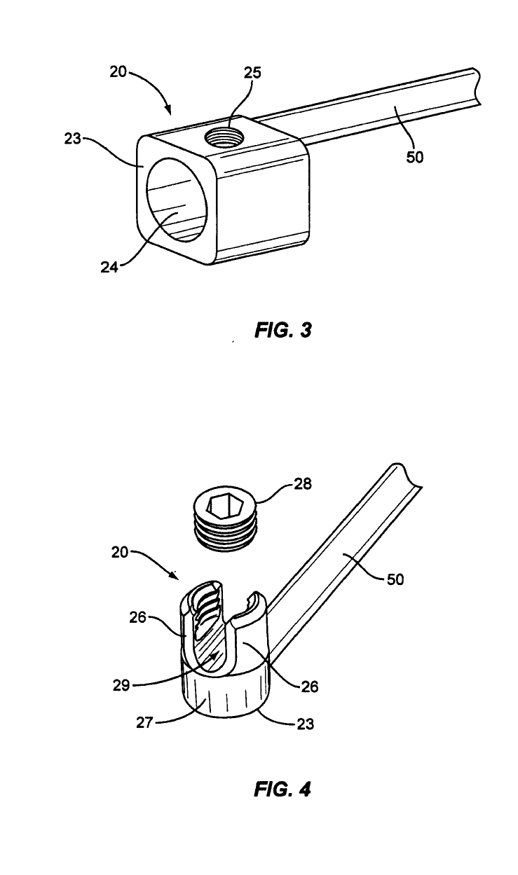

The body 23 of the first connector 20 may also extend around the elongated member 111 as illustrated in FIG. 3. An aperture 24 sized to receive the elongated member 111 extends through the body 23 along a first axis. A second aperture 25 may extend into the body 23 along a second axis and be in communication with the aperture 24. The second aperture 25 is sized to receive a fastener (not illustrated) such as a set screw that extends into the aperture 24 and contacts against the elongated member 111 to maintain the connection.

FIG. 4 includes the first connector 20 with a U-shaped body 23 that includes a pair of opposing arms 26 that extend outward from a base 27. The arms 26 and base 27 forms a rigid channel 29 sized to receive the elongated member 111. A fastener 28 is sized to fit within the channel 29 and capture the elongated member 111. FIG. 4 includes the fastener 28 with exterior threads that engage with threads along the interior of the arms 26. The fastener 26 may also be configured to extend around the exterior of the arms 26. The fastener 26 includes a threaded aperture that engages with threads on the exterior of the arms 26.

The second connector 30 is configured to connect with the deformable elongated member 121. FIG. 5 includes the second connector 30 with a rigid body 31 with a base 32 and opposing arms 33 that form a channel 34 sized to receive the elongated member 121. A fastener 35 is configured to connect with the arms 33 and capture the elongated member 121. The fastener 35 may include exterior threads that engage with interior threads on the arms 33 as illustrated in FIG. 5, or may include a threaded aperture to engage with threads on the exterior of arms 33. This second connector 30 is similar to the first connector 20 of FIG. 4.

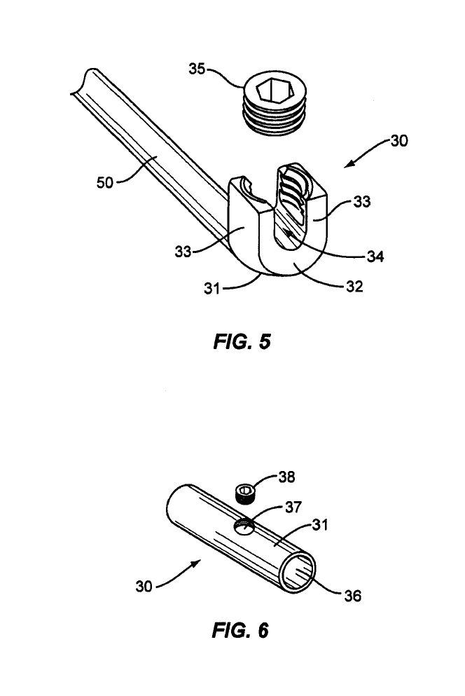

FIG. 6 includes the second connector 30 with an aperture 36 that extends through the body 31 along a first axis and is sized to receive the elongated member 121. The length of the body 31 may vary depending upon the context of use. A second aperture 37 may extend into the body 31 along a second axis and lead into the aperture 36. A fastener 38 engages within the second aperture 37 and includes a length to extend into the aperture 36 to contact against the elongated member 121 and form the connection. This second connector 30 is similar to the first connector 20 of FIG. 3.

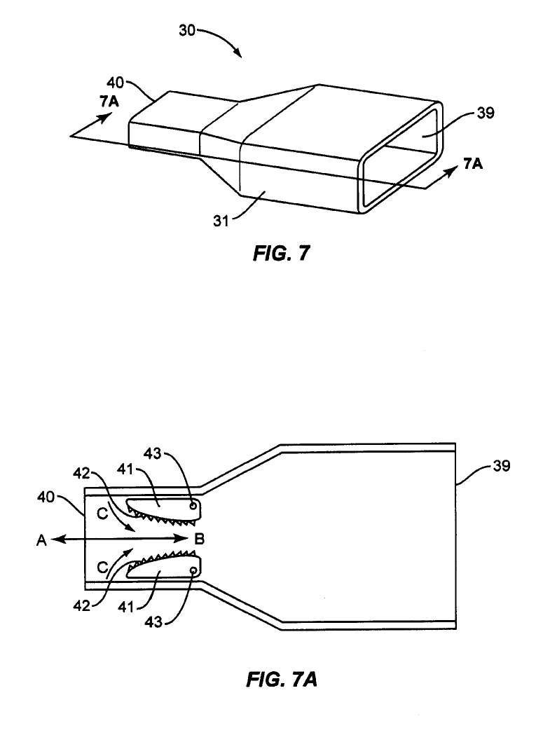

The non-fusion system 120 may include the elongated member 121 being under tension to apply a corrective force to the connected vertebral members 200. The second connector 30 may be configured to accommodate for maintaining the tension. FIGS. 7 and 7A illustrate a connector 30 with a body 31 with a first end 39 and a second end 40. The first end 39 faces away from the first connector 20. The body 31 may include a funnel-shape with the first end 39 being wider than the second end 40 to facilitate insertion of the elongated member 121 into the first end 39 and through the body 31 to the second end 40. Offsetting cam members 41 are attached to the body 31 at pivots 43. The pivots are positioned on the cam members 41 in closer proximity to the first end 39 than the second end 40. The surfaces of the cam members 41 include teeth 42. The cam members 41 are shaped and configured to allow insertion of the elongated member 121 in the direction of arrow A in FIG. 7A. During insertion of the elongated member 121, the cam members 41 pivot outward away from each other and a longitudinal axis to increase a space between the members 41 to allow insertion of the elongated member 121. Once inserted, the cam members 41 are in contact with the elongated member 121. The tension causes the cam members 41 to pivot inward in the direction indicated by arrows C towards the longitudinal axis to prevent the elongated member 121 from moving in the direction of arrow B.

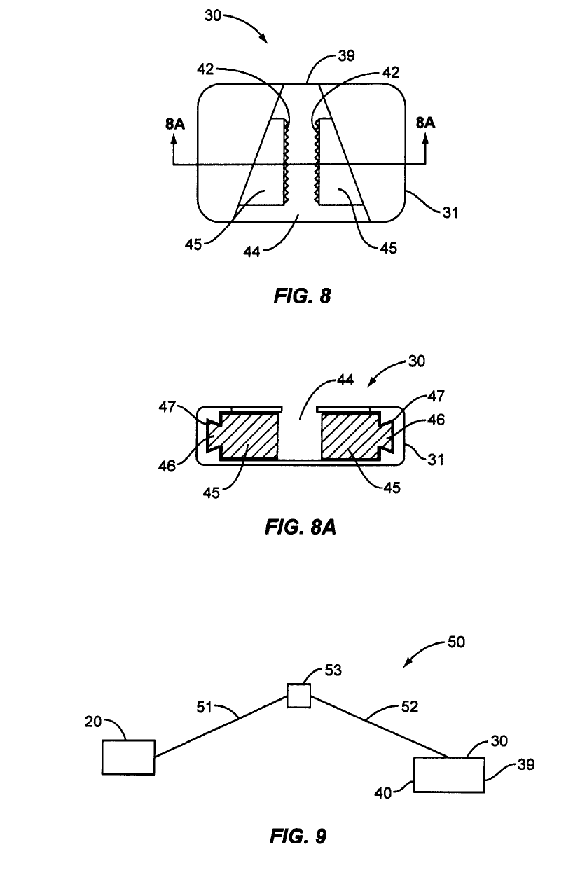

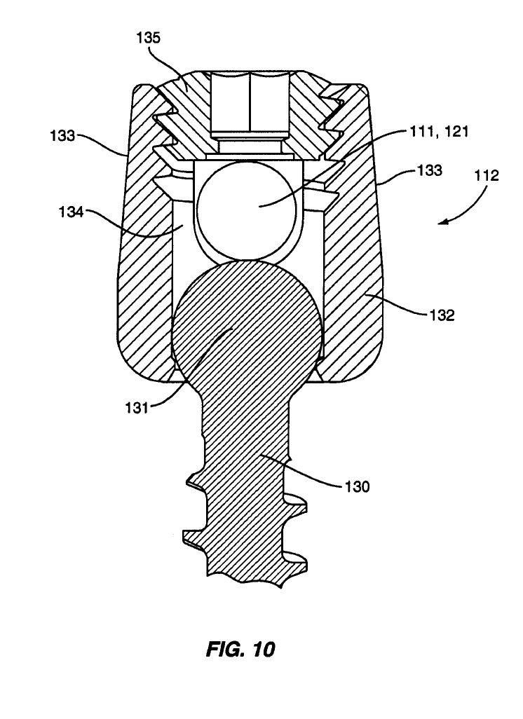

Another second connector 30 that maintains tension on the elongated member is illustrated in FIGS. 8 and 8A. The connector 30 includes a body 31 with a tapered opening 44 that extends between the first and second ends 39, 40 with the first end 39 facing away from the first connector 20. A pair of wedges 45 are slidably received on opposite sides of the tapered opening 44 for engaging the elongated member 121. The wedges 45 may include a substantially rectangular shape, or may include a tapered width that is wider towards the second end 40 and narrower towards the first end 39. Each of the wedges 45 is keyed and includes an extension 46 that mates within a slot 47 in the body 31. As illustrated in FIG. 8A, the extensions 46 may include a dovetail shape that fit within the corresponding dovetail slots 47. The wedges 45 can slide along the opening 44 between an open position away from the first end 39 with a distance between the inner sides of the wedges 45 being larger than the width of the elongated member 121, and a closed positioned in closer proximity to the first end 39 with the distance less than the width of the elongated member 121. In the closed position, the wedges 45 contact against and hold the elongated member 121. Teeth 42 may be positioned along the inner sides of the wedges 45 to facilitate the hold. The connector 30 may include both wedges 45 being movable along the opening 44, or just a single wedge 45 may be movable along the opening 44 with the other wedge 45 remaining stationary.

In use, the elongated member 121 may be threaded or laid/placed into the connector from the top down into the opening 44 from the first side 39 towards the second side 40. Movement of the elongated member 121 into the opening 44 in this direction moves the wedges 45 away from the first side 39 to the open position. The elongated member 121 may then be tensioned to an appropriate amount. The wedges 45 contact against the elongated member 121 and move towards the first end 39 to the closed position to prevent the elongated member 121 from escaping from the connector 30 and also maintain the tension.

Various types of connectors are disclosed in U.S. patent application Ser. No. 11/842,693 which is herein incorporated by reference in its entirety.

The bridge 50 extends between and connects the first and second connectors 20, 30. The bridge 50 functions to position the first and second connectors 20, 30 to connect to the elongated member 111 and elongated member 121 respectively. The bridge 50 may be formed from a single section as illustrated in FIG. 1, or as multiple sections 51, 52 as illustrated in FIG. 9. A connector 53 may provide for positioning the sections 51 and connectors 20, 30 at various angular positions. In one embodiment, each section 51 includes a flattened end with a central aperture. The flattened ends seat together and the apertures align to receive a fastener to fix the angular position. The flattened ends may also include splines to further control the angular position.

The bridge 50 may connect to the connectors 20, 30 at various positions. FIG. 1 includes the bridge 50 extending outward from the inner surfaces of each connector 20, 30. The bridge 50 may also extend outward from other areas of the connectors 20, 30.

The bridge 50 may be spaced away from an axis that extends through the first and second connectors 20, 30. As illustrated in FIGS. 1 and 2, the bridge 50 is positioned away from the axis L such that it does not interfere with the connection to the elongated member 111 or elongated member 121.

The bridge 50 may also include one or more extensions 55 that extend outward from a main section 54 as illustrated in FIG. 2. The extensions 55 are shaped to position the connectors 20, 30 to receive the elongated member 111 and elongated member 121. FIG. 2 includes the extensions 55 extending outward perpendicularly to the main section 54. When implanted into the patient, the bridge 50 may be at various positions relative to the vertebral members 200. FIG. 2 illustrates the bridge 50 spaced away from the vertebral members 200. The linkage 10 may also be rotated to various positions to position the bridge 50 against the vertebral members 200.

The bridge 50 may also be shaped to accommodate connection between overlapping or non-overlapping systems 110, 120. In one embodiment as illustrated in FIG. 1, the bridge 50 positions the connectors 20, 30 for accommodating the systems 110, 120 in an end-to-end orientation. The bridge 50 is shaped for the axis L to extend through each of the connectors 20, 30. The bridge 50 may also be configured to position the connectors 20, 30 in a laterally-offset position to accommodate overlapping systems 110, 120. This may include the bridge 50 with a particular shape for placement of the connectors 20, 30, or may include the connectors 20, 30 oriented relative to the bridge 50 at various angles to accommodate the systems 110, 120.

The elongated member 111 may be a rod constructed from a variety of surgical grade materials. These include metals such as stainless steels, cobalt-chrome, titanium, and shape memory alloys. Non-metallic elongated members, including polymer elongated members made from materials such as polyetheretherketone (PEEK) and UHMWPE, are also contemplated.

The elongated member 121 may include but is not limited to articulated rods, cables, artificial or synthetic strands, flexible rods, and springs. The elongated member 121 may include an inner core with an outer sheath. The inner core and outer sheath may be made of a braided polymer such as polyester, polypropylene, or polyethylene. In one specific embodiment, the inner core and outer sheath are both made of polyethylene with the inner core being braided for strength and the outer sheath being braided for abrasion resistance. In one embodiment with the elongated member 121 being a strand, the strand may be manufactured from a variety of materials, including, but not limited to, conventional biocompatible implant alloys such as titanium, stainless steel, cobalt-chrome alloys, or even shape memory alloys and materials such as nickel-titanium.

Anchors 112 connect the elongated member 111 and elongated member 121 to the vertebral members 200. FIG. 10 illustrates one embodiment of an anchor 112 for connecting the elongated member 111 to the vertebral members 200. The anchor 112 includes a screw with a shaft 130 that mounts to the vertebral member 200 and a substantially spherical head 131. A saddle 132 is movably mounted to the head 131 and includes opposing arms 133 that form an interior space 134 sized to receive the elongated member 111 or elongated member 121. A set screw 135 is threaded to the arms 133 to capture the elongated member 111 or elongated member 121 within the interior space 134. The saddle 132 is pivotally and rotatably connected to the head 131. This connection provides multi-axial movement of the elongated member 111 or elongated member 121 relative to the vertebral member 200 to which the shaft 130 is attached. Other anchors 112 may include a fixed connection in which the saddle 132 is fixedly connected to the shaft 130 and head 131. The anchors 112 may also include hooks, pegs, spikes, and other structures to connect to the vertebral members 200. Each of the anchors 112 along the fusion system 110 and non-fusion system 120 may be the same or different types of anchors 112 may be used at different locations along the length of the spine.

The elongated member 111 may extend completely or partially through the first connector 20. When partially extending through, the end of the elongated member is positioned within the body between the opposing ends. Likewise, the elongated member 121 may extend completely or partially through the second connector 30. The end of the elongated member 121 may be positioned inward of the second end 40 when extending completely through the connector 30, or positioned between the first and second ends 39, 40 when positioned partially through the second connector 30.

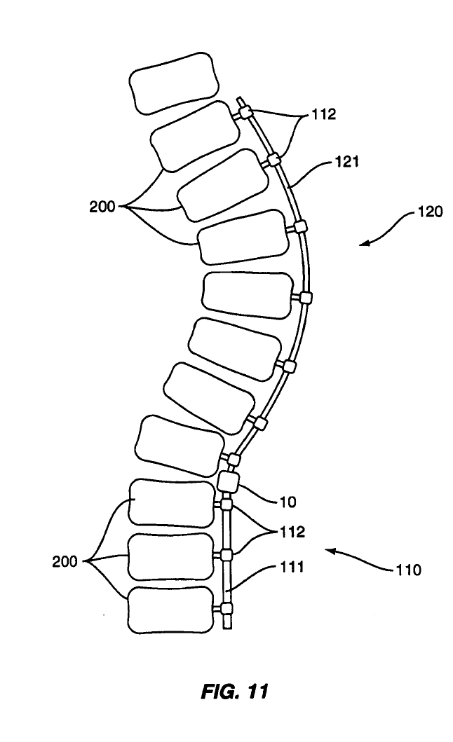

FIG. 11 is a schematic view of the linkage 10 connecting together the elongated member 111 of the fusion system 110. The fusion system 110 includes a elongated member 111 connected through anchors 112 to a first level of vertebral members 200. The non-fusion system 120 includes an elongated member 121 connected through anchors 112 to a second level of vertebral members 200. The linkage 10 connects the elongated member 111 and elongated member 121 in an end-to-end and non-overlapping orientation.

The fusion system 110 immobilizes the vertebral members 200 along the first level. The fusion system 110 is used in combination with a bone graft between the transverse processes or other vertebral protrusions or surfaces. The bone graft may rely on supplementary bone tissue and bone growth stimulators in conjunction with the body's natural bone growth processes to literally fuse vertebral members to one another.

The non-fusion system 120 is connected to the second level of vertebral members 200. In this embodiment, the spine includes a scoliotic curve with the curve being offset a distance from its correct alignment in the coronal plane. The spine is deformed laterally so that the axes of the vertebral members 200 are displaced from the sagittal plane passing through a centerline of the patient. In the area of the lateral deformity, the elongated member 121 is connected with anchors 112 to a convex side of the vertebral members 200. The elongated member 121 applies a compressive force to the convex side of the vertebral members 200 to reduce and/or eliminate the spinal deformity.

In use, the linkage 10 may be attached to one or both of the elongated member 111 and elongated member 121 either before or after being implanted into the patient. When implanted after insertion, the elongated member 111 is secured within anchors 112 along the first level of vertebral members 200, and the elongated member 121 is positioned within anchors 112 along the second level of vertebral members. The linkage 10 is secured to the elongated member 111 and the elongated member 121. The linkage 10 may contact the elongated member 111 and elongated member 121 at various locations. As illustrated in FIG. 1, the first connector 20 connects to the elongated member 111 inward from the outer-most anchor 112. Other locations may also include a connection at the axial end of the elongated member 111 (i.e., between the outer-most anchor 112 and the end of the elongated member 111). The second connector 30 may also include the same positioning relative to the elongated member 121.

Prior to being secured to the elongated member 121, an appropriate amount of tension is placed on the elongated member 121. This may include the elongated member 121 being threaded through the second connector 121 and pulled outward away from the second end 40. Once the proper amount of tension is applied, the second connector 30 may be secured to maintain the tension and prevent the elongated member 121 from moving.

It should be understood that the spinal deformity depicted in FIG. 11 is but one of many types of spinal deformities that can be addressed by the devices and techniques of the present application. Most commonly the devices and methods are expected to be used in the setting or spinal surgery where risk of junctional failure or deformity exists. This could entail stabilization of junctional zones above or below fusion areas or surgical settings of revision surgery. The devices and methods can be used for correction of the thoracic curve as an isolated curve, or the lumbar curve as an isolated curve. The devices may further be used in combination with the shortening of the opposite side of the vertebral member 200.

The fusion systems 110 described above each include an elongated member 111 that may include but is not limited to rods, cables, and wires.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A linkage to connect an elongated member of a fusion system with an elongated member of a non-fusion system, the linkage comprising:
a bridge with a first end and a second end;
a first connector positioned at the first end of the bridge to connect with the elongated member of the fusion system, the first connector at least partially extending around and contacting against an outer surface of the elongated member of the fusion system;
a second connector positioned at the second end of the bridge to connect with the elongated member of the non-fusion system, the second connector including opposing contact surfaces that contact against an outer surface of the elongated member of the non-fusion system;
the first and second connectors being aligned along a longitudinal axis to position the elongated member of the fusion system and the elongated member of the non-fusion system in an end-to-end and non-overlapping orientation;
the bridge being spaced to a side of each of the first and second connectors and away from the longitudinal axis.

2. The linkage of claim 1, wherein the bridge is straight.

3. The linkage of claim 1, wherein the bridge includes first and second sections pivotally connected together at a connector.

4. The linkage of claim 1, wherein the second connector includes first and second opposing contact surfaces positioned to contact against the elongated member of the non-fusion system, at least one of the contact surfaces is adjustable to change a distance between the contact surfaces to maintain a tension on the elongated member of the non-fusion system by preventing the elongated member from moving away from the first connector.

5. The linkage of claim 4, wherein the second connector includes a body with a tapered opening with one of the contact surfaces positioned on a wedge that is keyed to the body and movable along the tapered opening between a first position spaced a first distance away from the opposing contact surface and a second position spaced a second farther distance away from the opposing contact surface.

6. The linkage of claim 4, wherein the second connector includes a body with an opening and a pair of pivoting cam members positioned on opposing sides of the opening, the cam members each being pivotally connected to the body and movable between a first position spaced a first distance away from a longitudinal axis of the opening and a second position spaced a second farther distance away from the longitudinal axis of the opening.

7. The linkage of claim 1, wherein the bridge includes a main section and an extension that extends perpendicularly outward with one of the first and second connectors positioned on the extension.

8. A linkage to connect a rod of a fusion system with an elongated member of a non-fusion system, the linkage comprising:
an elongated bridge with a first end and a second end;
a first connector connected to the first end of the bridge to connect with the rod, the first connector comprising a receiver that at least partially extends around the rod and contacts against an outer surface of the rod;
a second connector connected to the second end of the bridge to connect with the elongated member, the second connector including a body with a first end that faces away from the first connector, a second end that faces towards the first connector, and an opening that extends between the first and second ends and is sized to receive the elongated member, the second connector further including first and second contact members movably connected to the body and positioned along the opening, the contact members movable between a first position with the contact members spaced a first distance apart to contact against the elongated member and a second position spaced a greater second distance apart to allow the elongated member to move through the opening.

9. The linkage of claim 8, wherein the contact members are positioned in closer proximity to the second end than to the first end of the body.

10. The linkage of claim 8, wherein the opening is tapered and includes a smaller width at the first end and a larger width at the second end.

11. The linkage of claim 8, wherein the first and second contact members each include a key that connects to the body to be movable along the tapered opening between a first position with a first distance between the contact members and a second position with a greater second distance between the contact members.

12. The linkage of claim 11, wherein each of the first and second contact members include a wedge shape with a tapered width.

13. The linkage of claim 8, wherein each of the contact members is pivotally connected to the body and movable between a first position spaced a first distance away from a center line of the opening and a second position spaced a second farther distance away from the center line of the opening.

14. The linkage of claim 8, wherein the first and second connectors are aligned along a longitudinal axis to position the rod and the elongated member in an end-to-end and non-overlapping orientation.

15. The linkage of claim 14, wherein the bridge is spaced to a side of each of the first and second connectors and away from the longitudinal axis.

16. A method of connecting an elongated member of a fusion system connected to a first series of vertebral members to an elongated member of a non-fusion system connected to a second series of vertebral members, the method comprising:
inserting the elongated member of the fusion system into a first connector of a linkage positioned at a first end of a bridge;
connecting the first connector to the elongated member;
inserting the elongated member of the non-fusion system into the second connector of the linkage positioned at a second end of the bridge; and
applying a tension force to the elongated member and connecting the second connector to the elongated member and maintaining the elongated member in tension.

17. The method of claim 16, further comprising connecting the first connector to an interior section of the rod and away from an end of the rod.

18. The method of claim 16, wherein inserting the elongated member of the non-fusion system into the second connector comprises sliding a contact member away from a first end of the second connector and away from a center line of an opening that extends through the first connector and increasing a width between the contact member and a second contact member positioned on an opposing side of the center line.

19. The method of claim 18, further comprising sliding the contact member towards the first end of the second connector and decreasing a width between the contact member and the opposing contact member and securing the elongated member.

20. The method of claim 16, further comprising positioning the linkage with the elongated members of the fusion and non-fusion systems in an end-to-end and non-overlapping orientation.
